(19) United States
(12) Patent Application Publication
(10) Pub. No.: US 2019/0313972 A1
(43) Pub. Date: Oct. 17, 2019

(54) USER TERMINAL APPARATUS AND DATA TRANSMISSION METHOD

(71) Applicants: OMRON CORPORATION, Kyoto-shi (JP); OMRON HEALTHCARE CO., LTD., Kyoto (JP)

(72) Inventors: Natsuko HORIGUCHI, Kyoto-shi (JP); Hiroshi NAKAJIMA, Kyoto-shi (JP); Tomohiro KUKITA, Kyoto (JP); Hirotaka WADA, Kyoto-shi (JP); Tamio UEDA, Osaka (JP)

(21) Appl. No.: 16/454,308

(22) Filed: Jun. 27, 2019

Related U.S. Application Data
(63) Continuation of application No. PCT/JP2017/044392, filed on Dec. 11, 2017.

(30) Foreign Application Priority Data
Jan. 4, 2017 (JP) 2017-000241

(51) Int. Cl. A61B 5/00; H01Q 1/27; A61B 5/021; G16H 10/65

(52) U.S. Cl. CPC A61B 5/681; G16H 10/65; A61B 5/021; H01Q 1/273

(57) ABSTRACT

According to one aspect, a user terminal apparatus includes a hardware processor and a memory connected to the hardware processor. The memory stores a plurality of blood pressure change patterns. The hardware processor is configured to acquire blood pressure data including a plurality of blood pressure values output from a blood pressure sensor configured to measure a blood pressure of a user, select a blood pressure change pattern corresponding to the blood pressure data from the plurality of blood pressure change patterns, and transmit transmission data including identification information identifying the selected blood pressure change pattern and part of the blood pressure data.

### CROSS-REFERENCE TO RELATED APPLICATIONS

This application is a Continuation Application of PCT Application No. PCT/JP2017/044392, filed Dec. 11, 2017 and based upon and claiming the benefit of priority from Japanese Patent Application No. 2017-000241, filed Jan. 4, 2017, the entire contents of all of which are incorporated herein by reference.

### FIELD

Embodiments of the present invention relate to a user terminal apparatus that measures biological information such as blood pressure.

### BACKGROUND

Measurement results of the biological information of a user are used in various scenes such as health care. For example, Jpn. Pat. Appln. KOKAI Publication No. 9-276238 discloses a technique of sensing a data pattern such as a respiratory pattern or blood pressure pattern from a person to be diagnosed and outputting an alert when the sensed data pattern is substantially identical to a predetermined data pattern.

In recent years, with advances in sensor technology, for example, a user terminal apparatus has been implemented which can perform measurement (for example, continuous measurement) of the blood pressure of a user by being worn on a wrist of the user. This user terminal apparatus can measure the blood pressure of a user without applying much burden on the user.

To continuously measure the blood pressure of a user is to generate a large amount of blood pressure data. In order to entirely save a large amount of blood pressure data, a large-capacity storage device is required. Entirely transmitting a large amount of blood pressure data to an external device for, for example, analysis will apply a large load to a communication path with the external device and consume a large amount of power.

### SUMMARY

A first aspect of the present invention is a user terminal apparatus including a blood pressure sensor configured to measure a blood pressure of a user and obtain blood pressure data including a plurality of blood pressure values, a storage unit configured to store a plurality of blood pressure change patterns, a selection unit configured to select a blood pressure change pattern corresponding to the blood pressure data from the plurality of blood pressure change patterns, and a transmission unit configured to transmit transmission data including identification information identifying the selected blood pressure change pattern and part of the blood pressure data.

According to the first aspect, when the blood pressure pattern obtained as a result of measurement by the blood pressure sensor matches the data of a blood pressure change pattern, transmission data including identification information identifying the blood pressure change pattern and part of the blood pressure data is transmitted to the external device. This allows the external device to acquire the blood pressure data without transmitting all the blood pressure data to the external device. The data of the identification information is much smaller than the blood pressure, and hence the amount of data transmitted can be reduced. That is, it is possible to reduce the power consumption associated with data transmission and the load applied to the communication path.

In a second aspect of the present invention, the selection unit is configured to calculate a degree of matching between the blood pressure data and each of the plurality of blood pressure change patterns, perform no selection when the highest degree of matching is less than a threshold, and select a blood pressure change pattern exhibiting the highest degree of matching from the plurality of blood pressure change patterns when the highest degree of matching exceeds the threshold.

According to the second aspect, when there is no blood pressure change pattern matching blood pressure data, blood pressure data is transmitted to an external device without being converted into a blood pressure change pattern. This allows the external device to obtain more accurate blood pressure data.

In a third aspect of the present invention, the user terminal apparatus further includes a generation unit configured to generate a blood pressure change pattern based on the blood pressure data, wherein the plurality of blood pressure change patterns include the generated blood pressure change pattern.

According to the third aspect, a blood pressure change pattern unique to a user is generated. This increases the possibility that blood pressure data will match a blood pressure change pattern and can further reduce the amount of data transmitted.

### BRIEF DESCRIPTION OF THE DRAWINGS

FIG. 1 is a block diagram showing a user terminal apparatus according to one embodiment of the present invention;

FIG. 2 is a perspective view showing an example of the outer appearance of the user terminal apparatus shown in FIG. 1;

FIG. 3 is a block diagram showing an example of the arrangement of a biological information management system including the user terminal apparatus shown in FIG. 1;

FIG. 4 is a graph showing an example of blood pressure variation; and

FIG. 5 is a flowchart showing an example of a procedure in which the user terminal apparatus shown in FIG. 1 transmits blood pressure data.

### DETAILED DESCRIPTION

According to an embodiment of the present invention, there is provided a user terminal apparatus and a data transmission method that can reduce the amount of data transmitted to an external device.

Hereinafter, embodiments of the present invention will be described with reference to the accompanying drawings.

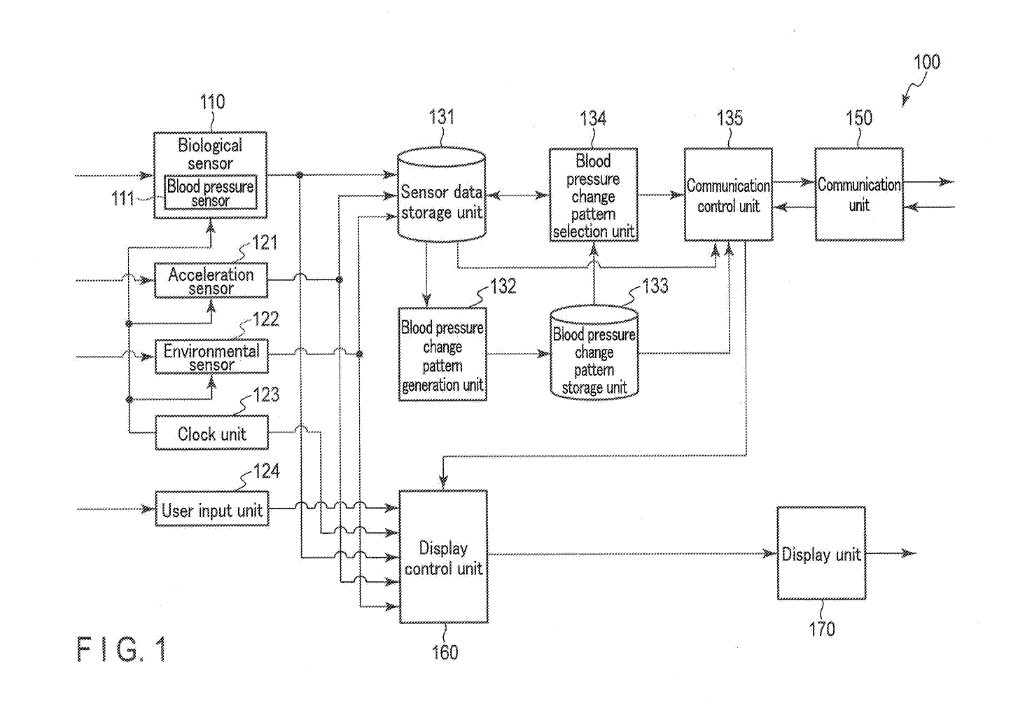

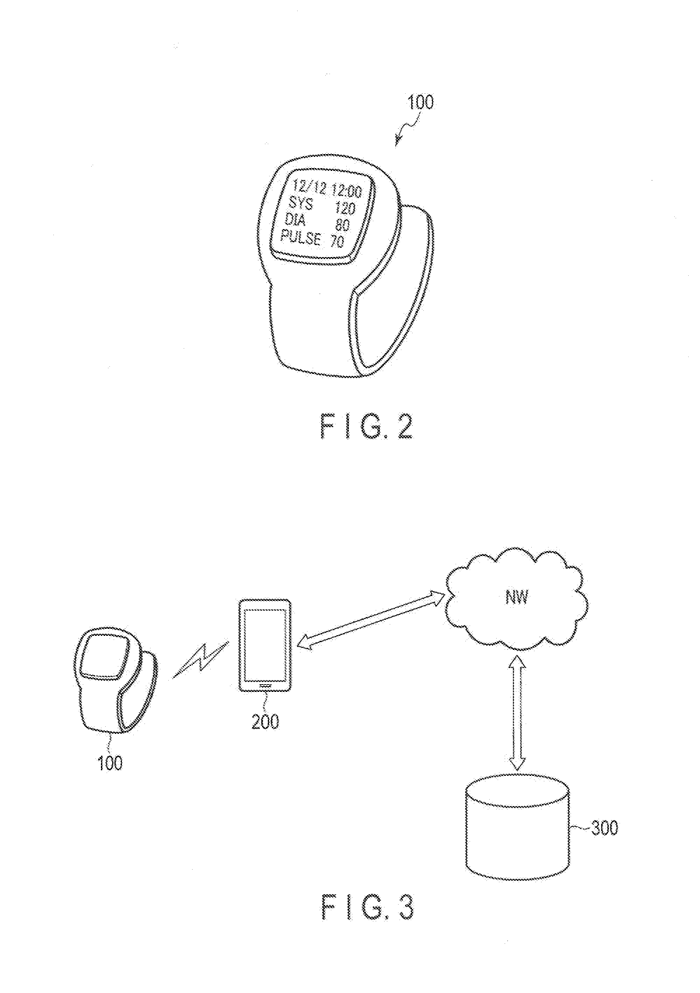

FIG. 1 schematically shows a user terminal apparatus 100 according to one embodiment of the present invention. The user terminal apparatus 100 shown in FIG. 1 may be a wearable device such as a wristwatch-type wearable device illustrated in FIG. 2. The user terminal apparatus 100 can measure biological information such as the systolic blood pressure (SBP), diastolic blood pressure (DBP), and heart rate of the user who wears the user terminal apparatus 100. The user terminal apparatus 100 can display a measurement result together with information displayed on a general clock, such as a date and time.

As shown in FIG. 3, the user terminal apparatus 100 may be connected to a smart device 200. Typically, the smart device 200 may be a portable device such as a smartphone or tablet. The smart device 200 can display biological data transmitted by the user terminal apparatus 100 in a graphical form and transmit the biological data to a server 300 via a network NW. An application that manages biological data may be installed in the smart device 200. Note that the user terminal apparatus 100 may be connected to the server 300 via the network NW without via the smart device 200.

The server 300 stores biological data transmitted from the user terminal apparatus 100 or the smart device 200. The server 300 may transmit the biological data in response to an access made from a personal computer (PC) installed in a medical institution to use the biological data, for example, for medical care guidance or diagnosis of the user. The server 300 can analyze a factor that causes a change in user state based on the stored biological data and generate an improvement proposal for setting the user state to a user state defined as better. The server 300 then can transmit the factor analysis result and the improvement proposal to the user terminal apparatus 100 or the smart device 200 to make the user browse them.

Referring to FIG. 1, the user terminal apparatus 100 includes a biological sensor 110, an acceleration sensor 121, an environmental sensor 122, a clock unit 123, a user input unit 124, a sensor data storage unit 131, a blood pressure change pattern generation unit 132, a blood pressure change pattern storage unit 133, a blood pressure change pattern selection unit 134, a communication control unit 135, a communication unit 150, a display control unit 160, and a display unit 170.

The biological sensor 110 measures the biological information of the user to obtains biological data, and sends the biological data to the sensor data storage unit 131 and the display control unit 160. The biological sensor 110 includes at least a blood pressure sensor 111 that measures the blood pressure of the user to obtain blood pressure data. That is, biological data includes at least blood pressure data. In addition, the biological data may include electrocardiogram data, heart rate data, pulse wave data, pulsation data, and body temperature data. Each biological data can be associated with measurement time set based on the time information received from the clock unit 123.

According to this embodiment, the blood pressure sensor 111 includes a continuous blood pressure sensor. The continuous blood pressure sensor is a blood pressure sensor capable of continuously measuring blood pressures per heartbeat (for example, a systolic blood pressure and a diastolic blood pressure). The continuous blood pressure sensor can be based on a technique of measuring a pulse transmit time (PTT) and estimating a blood pressure from the measured pulse transmit time, a tonometry method, or another technique. Blood pressure data includes, but is not limited to, a systolic blood pressure value and a diastolic blood pressure value per heartbeat.

The blood pressure sensor 111 may further include a noncontinuous blood pressure sensor. A noncontinuous blood pressure sensor includes, as an example, a blood pressure sensor being based on an oscillometric method of measuring a blood pressure by using cuff as a pressure sensor. The noncontinuous blood pressure sensor (an oscillometric blood pressure sensor in particular) tends to have higher measurement accuracy than a continuous blood pressure sensor. Accordingly, the blood pressure sensor 111 is triggered by, for example, satisfying a certain condition (for example, when the blood pressure data of the user measured by the continuous blood pressure sensor indicates a predetermined high risk state) to operate the noncontinuous blood pressure sensor in place of the continuous blood pressure sensor, thereby measuring blood pressure data with high accuracy.

The acceleration sensor 121 detects the acceleration applied to the acceleration sensor 121 to obtain three-axis acceleration data. This acceleration data can be used to estimate the activity state (posture and/or action) of the user wearing the user terminal apparatus 100. The acceleration sensor 121 sends the acceleration data to the sensor data storage unit 131 and the display control unit 160. The acceleration data can be associated with the measurement time set based on the time information received from the clock unit 123.

The environmental sensor 122 measures environmental information around the user terminal apparatus 100 to obtain environmental data, and sends the environmental data to the sensor data storage unit 131 and the display control unit 160. The environmental data may include temperature data, humidity data, and atmospheric pressure data. Each environmental data can be associated with the measurement time set based on the time information received from the clock unit 123.

The clock unit 123 generates time information representing the current time in a predetermined cycle, and sends the time information to the biological sensor 110, the acceleration sensor 121, the environmental sensor 122, and the display control unit 160. Time information can be used as the time of measurement of biological data by the biological sensor 110, the time of measurement of acceleration data by the acceleration sensor 121, and the time of measurement of environmental data by the environmental sensor 122. The clock unit 123 may include a calendar function. That is, the clock unit 123 may generate date information representing the current date and send the date information to the display control unit 160.

The user input unit 124 includes, for example, buttons and dials for the reception of user inputs. Alternatively, a combination of the user input unit 124 and the display unit 170 (to be described later) may be implemented by using a touch screen. A user input is, for example, the operation of controlling the display screen of the display unit 170.

The sensor data storage unit 131 stores the biological data output from the biological sensor 110, the acceleration data output from the acceleration sensor 121, and the environmental data output from the environmental sensor 122. As described above, biological data includes blood pressure data.

A blood pressure change pattern generation unit 132 acquires blood pressure data from the sensor data storage unit 131 and generates a blood pressure change pattern based on the blood pressure data. The blood pressure change pattern is a pattern indicating a change (temporal variation) in blood pressure value in a unit period. More specifically, a blood pressure change pattern corresponds to a waveform and is expressed by a relative blood pressure value. In other words, in order to reproduce blood pressure data by using a blood pressure change pattern, at least one data value is required. A unit period is, but is not limited to, 1 min, 1 hr, or 1 day. A blood pressure value change indicates a different pattern for each user. A blood pressure value change indicates a different pattern for each temporal condition such as each season, day, or a biological clock (circadian rhythm). A blood pressure value change indicates a different pattern in accordance with a user state (for example, awaking, standing, seating, exercising, stress, movement from indoor to outdoor, or bathing). The blood pressure change pattern generation unit 132 can generate a blood pressure change pattern for each of many conditions. The communication unit 150 (to be describe later) transmits the data of the blood pressure change pattern generated by the blood pressure change pattern generation unit 132 to an external device such as the smart device 200 or the server 300 shown in FIG. 2.

This allows both the user terminal apparatus 100 and the external device to hold the data of the blood pressure change pattern. The blood pressure change pattern may be provided in any format, for example, a data set or function.

A blood pressure change pattern storage unit 133 stores a plurality of blood pressure change patterns generated by the blood pressure change pattern generation unit 132. Identification information is given to each blood pressure change pattern. The blood pressure change pattern storage unit 133 may store a plurality of blood pressure change patterns prepared in advance. These blood pressure change patterns can be generated based on the blood pressure data obtained from many subjects. In this case, the user terminal apparatus 100 may or may not include the blood pressure change pattern generation unit 132. The blood pressure change pattern generated by the blood pressure change pattern generation unit 132 is a blood pressure change pattern unique to the user, and is likely to be selected by the blood pressure change pattern selection unit 134 (to be described later). Any blood pressure change pattern that is selected at a low frequency may be deleted from the blood pressure change pattern storage unit 133.

The blood pressure change pattern selection unit 134 selects a blood pressure change pattern corresponding to blood pressure data in a unit period extracted from blood pressure data among a plurality of blood pressure change patterns stored in the blood pressure change pattern storage unit 133, and sends identification information identifying the selected blood pressure change pattern to the communication control unit 135. For example, the blood pressure change pattern selection unit 134 extracts blood pressure data in a unit period from blood pressure data, calculates the degree of matching between the extracted blood pressure data in a unit period and each of a plurality of blood pressure change patterns, and selects a blood pressure change pattern exhibiting the highest degree of matching. If the highest degree of matching is less than a threshold, no blood pressure change pattern may be selected. In this case, the blood pressure data in the unit period is not converted into a blood pressure change pattern, and can be transmitted to an external device. The blood pressure change pattern selection unit 134 may further send identification information identifying the selected blood pressure change pattern to the sensor data storage unit 131. This makes it possible to replace the blood pressure data in the unit period with blood pressure data including the identification information and part of the blood pressure data. Because the data amount of the identification information is much smaller than blood pressure data, the amount of data stored in the sensor data storage unit 131 can be reduced.

The communication control unit 135 controls the communication unit 150. The communication control unit 135 generates transmission data including the identification information received from the blood pressure change pattern selection unit 134 and part of corresponding blood pressure data in a unit period, and sends the generated transmission data to the communication unit 150. Part of the blood pressure data in the unit period is, for example, one blood pressure value (for example, the first blood pressure value in the unit period). The communication control unit 135 can further generate transmission data including the data of the blood pressure change pattern generated by the blood pressure change pattern generation unit 132 and identification information identifying the blood pressure change pattern.

The communication unit 150 exchanges data with an external device such as the smart device 200 or the server 300 shown in FIG. 2. The communication unit 150 performs one or both of wireless communication and wired communication. For example, the communication unit 150 performs short-range wireless communication such as Bluetooth® with the smart device 200. The communication unit 150 receives transmission data from the communication control unit 135, and transmits the data to the external device. The communication unit 150 receives data from the external device and forwards the received data to the communication control unit 135.

The display control unit 160 controls the display unit 170. More specifically, the display control unit 160 generates screen data and sends it to the display unit 170. The display control unit 160 can generate screen data based on biological data from the biological sensor 110, acceleration data from the acceleration sensor 121, environmental data from the environmental sensor 122, time information and date information from the clock unit 123, a factor analysis result and an improvement proposal from the communication control unit 135, and the like. The display control unit 160 may select information to be used to generate screen data in accordance with a user input corresponding to an operation concerning control of the display screen of the display unit 170.

The display unit 170 is, for example, a liquid crystal display or organic electroluminescence (EL) display. The display unit 170 can notify the user of various information by displaying screen data from the display control unit 160. More specifically, the display unit 170 may display biological information (for example, a blood pressure, electrocardiogram, heart rate, pulse wave, pulse rate, and body temperature), acceleration data, activity amount information (for example, the number of steps counted based on acceleration data and a consumed calorie), sleep information (for example, a sleep time), environmental information (for example, a temperature, humidity, and atmospheric pressure), a factor analysis result, an improvement proposal, the current time, a calendar, and the like.

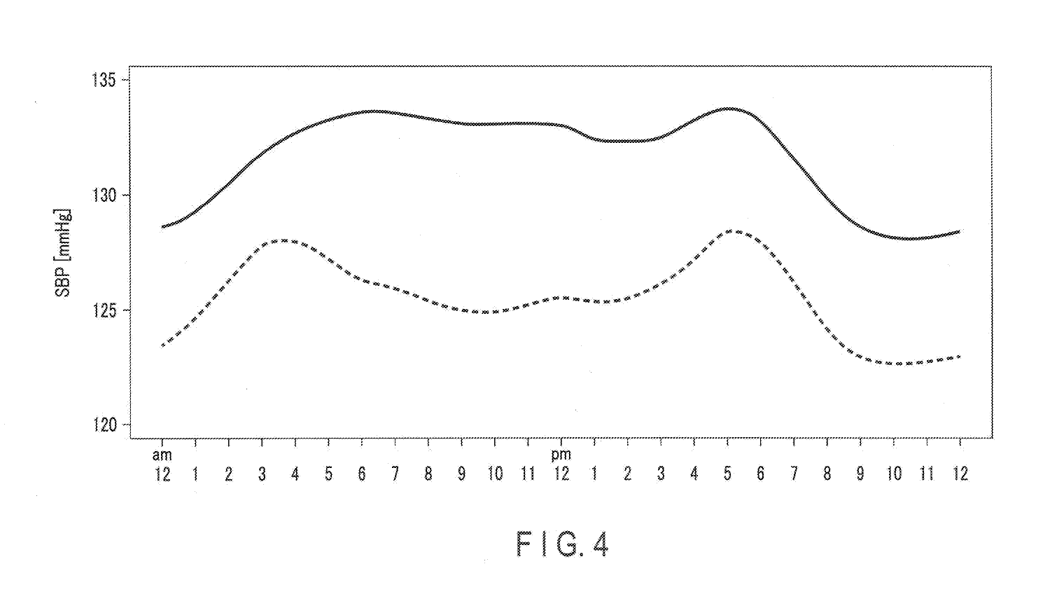

FIG. 4 shows an example of blood pressure variation. Referring to FIG. 4, the solid line represents variations in systolic blood pressure value of a given male user per day, and the broken line represents variations in systolic blood pressure value of a given female user per day. The waveforms shown in FIG. 4 can be generated as blood pressure change patterns.

The unit time may be changed as needed. In this case, the blood pressure change pattern storage unit 133 holds blood pressure change patterns in different unit times. For example, the unit time can be changed in accordance with the state of the user. When, for example, a user with a sleep apnea syndrome uses the user terminal apparatus 100, detailed blood pressure data is required to be obtained while the user is sleeping. Accordingly, for example, the unit time is reduced to obtain detailed blood pressure data while the user is sleeping, whereas the unit time is increased to further reduce the amount of data transmitted while the user is awake. Whether the user is sleeping can be detected by using a sensor like the acceleration sensor 121. In another case, the unit time can be changed in accordance with a time zone. For example, blood pressure values in time zones before and after bedtime and time zones before and after wake-up are considered important in health care. Accordingly, the unit time in the time zones from 22:00 to 24:00 and in the time zone from 5:00 to 7:00 are reduced to obtain detailed blood pressure data. The unit times in other time zones are increased to further reduce the amount of transmission data.

The user terminal apparatus 100 includes, for example, a central processing unit (CPU) and a memory as hardware. The memory includes a read only memory (ROM), random access memory (RAM), and secondary storage device. Various types of functions of the user terminal apparatus 100 can be implemented by causing the CPU to read out a program from the ROM or secondary storage device into the RAM and executing the program. As the secondary storage device, for example, a semiconductor memory or hard disk drive (HDD) can be used. The secondary storage device includes the sensor data storage unit 131 and the blood pressure change pattern storage unit 133. Note that some or all of the functions of the user terminal apparatus 100 may be implemented by hardware such as IC chips.

An operation of the user terminal apparatus 100 will be described next.

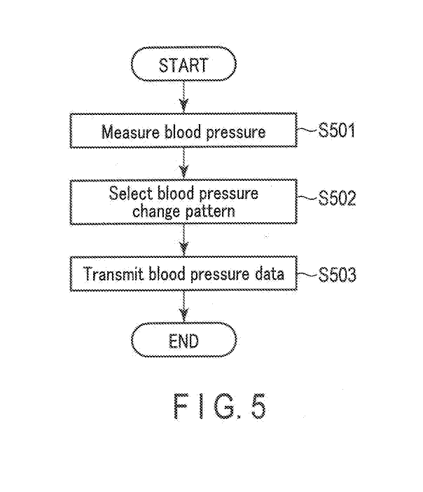

FIG. 5 shows an example of a procedure in which the user terminal apparatus 100 transmits blood pressure data.

Consider, for the sake of simplicity, a case in which blood pressure data in a unit time is acquired by using the blood pressure sensor, and the blood pressure data is transmitted to an external device. In step S501 in FIG. 5, the blood pressure sensor 111 continuously measures the blood pressure of the user, and obtains blood pressure data including a plurality of blood pressure values. Blood pressure data includes, for example, time-series data associated with a systolic blood pressure value, a diastolic blood pressure value, or both of them.

In step S502, the blood pressure change pattern selection unit 134 selects a blood pressure change pattern corresponding to blood pressure data from a plurality of blood pressure change patterns stored in the blood pressure change pattern storage unit 133. In step S503, the communication unit 150 transmits transmission data including identification information identifying the blood pressure change pattern selected by the blood pressure change pattern selection unit 134 and one data value of the blood pressure data.

As described above, in this embodiment, when the blood pressure data obtained as a result of continuous measurement by the blood pressure sensor matches the data of a blood pressure change pattern, the user terminal apparatus transmits transmission data including identification information identifying the blood pressure change pattern and part of the blood pressure data to the external device. This makes it possible for the external device to acquire blood pressure data without transmitting all the blood pressure data to the external device. Because the data amount of identification information is much smaller than blood pressure data, the amount of data transmitted can be reduced. That is, this can reduce the power consumption associated with data transmission and the load applied to the communication path.

In another embodiment, a blood pressure sensor includes a noncontinuous blood pressure sensor without including any continuous blood pressure sensor. Even in this case, the blood pressure sensor measures blood pressures at a plurality of time points, and hence blood pressure data includes a plurality of blood pressure values. In another embodiment, therefore, processing blood pressure data in the same manner as in the above embodiment can obtain the same effects as those of the above embodiment.

Note that the present invention is not limited to each embodiment described above, and elements can be modified and embodied in the execution stage within the spirit and scope of the invention. In addition, various inventions can be formed by proper combinations of a plurality of elements disclosed in the above embodiments. For example, several elements may be omitted from all the elements disclosed in the above embodiments. Furthermore, elements in different embodiments may be properly combined.

Although some or all of the embodiments described above can be described as in the following supplementary note as well as in the scope of claims, this is not exhaustive.

(Supplementary Note 1)

A user terminal apparatus comprising:

a hardware processor; and

a memory connected to the hardware processor,

wherein the memory includes a storage unit configured to store a plurality of blood pressure change patterns, and

the hardware processor is configured to:

acquire blood pressure data including a plurality of blood pressure values output from a blood pressure sensor configured to measure a blood pressure of a user;

select a blood pressure change pattern corresponding to the blood pressure data from the plurality of blood pressure change patterns; and

transmit transmission data including identification information identifying the selected blood pressure change pattern and part of the blood pressure data.

(Supplementary Note 2)

A data transmission method comprising:

obtaining, by a hardware processor, blood pressure data including a plurality of blood pressure values output from a blood pressure sensor configured to measure a blood pressure of a user;

selecting, by a hardware processor, a blood pressure change pattern corresponding to the blood pressure data from a plurality of blood pressure change patterns; and

transmitting, by a hardware processor, transmission data including identification information identifying the selected blood pressure change pattern and part of the blood pressure data.

1. A user terminal apparatus comprising:
a hardware processor; and
a memory connected to the hardware processor,
wherein the memory is configured to store a plurality of blood pressure change patterns, and
the hardware processor is configured to:
acquire blood pressure data including a plurality of blood pressure values output from a blood pressure sensor configured to measure a blood pressure of a user;
select a blood pressure change pattern corresponding to the blood pressure data from the plurality of blood pressure change patterns; and
transmit transmission data including identification information identifying the selected blood pressure change pattern and part of the blood pressure data.

2. The user terminal apparatus according to claim 1, wherein the hardware processor is configured to calculate a degree of matching between the blood pressure data and each of the plurality of blood pressure change patterns, perform no selection when the highest degree of matching is less than a threshold, and select a blood pressure change pattern exhibiting the highest degree of matching from the plurality of blood pressure change patterns when the highest degree of matching exceeds the threshold.

3. The user terminal apparatus according to claim 1, wherein the hardware processor is further configured to generate a blood pressure change pattern based on the blood pressure data, and
the plurality of blood pressure change patterns include the generated blood pressure change pattern.

4. A data transmission method comprising:
obtaining blood pressure data including a plurality of blood pressure values output from a blood pressure sensor configured to measure a blood pressure of a user;
selecting a blood pressure change pattern corresponding to the blood pressure data from the plurality of blood pressure change patterns; and
transmitting transmission data including identification information identifying the selected blood pressure change pattern and part of the blood pressure data.
